SEQUENCE LISTING (2 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, Mus musculus
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

(19) United States
(12) Patent Application Publication
(10) Pub. No.: US 2005/0153880 A1
(43) Pub. Date: Jul. 14, 2005

(54) Methods of treating or preventing ibd with il-18

(76) Inventors: Yukio Goto, Ibaraki (JP); Hideo Kikkawa, Ibaraki (JP); Mine Kinoshita, Ibaraki (JP)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/518,333

(22) PCT Filed: Jun. 5, 2003
(86) PCT No.: PCT/EP03/17744

(30) Foreign Application Priority Data
Jun. 27, 2002 (US) 60392176

(51) Int. Cl. A61K 38/17

(52) U.S. Cl. 514/12

(57) ABSTRACT

The present invention relates generally to the use of IL-18, also known as interferon-γ-inducing factor (IGIF), in the prevention and/or treatment of inflammatory bowel diseases.

### FIELD OF THE INVENTION

The present invention relates generally to the use of IL-18, also known as interferon-γ-inducing factor (IGIF), in the prevention and/or treatment of inflammatory bowel diseases.

### BACKGROUND OF THE INVENTION

IL-18 is a recently discovered novel cytokine. Active IL-18 contains 157 amino acid residues. It has potent biological activities, including induction of interferon-γ-production by T cells and splenocytes, enhancement of the killing activity of NK cells and promotion of the differentiation of naive CD4⁺T cells into Th1 cells. In addition, human IL-18 augments the production of GM-CSF and decreases the production of IL-10. IL-18 has been shown to have greater interferon-γ inducing capabilities than IL-12, and appears to have different receptors and utilize a distinct signal transduction pathway.

CD4⁺ T cells are the central regulatory elements of all immune responses. They are divided into two subsets, Th1 and Th2. Each subset is defined by its ability to secrete different cytokines. Interestingly, the most potent inducers for the differentiation are cytokines themselves. The development of Th2 cells from naive precursors is induced by IL-4. Prior to the discovery of IL-18, IL-12 was thought of as the principal Th1 inducing cytokine. IL-18 is also a Th1 inducing cytokine and is more potent than IL-12 in stimulating the production of interferon-γ.

Th1 cells secrete IL-2, interferon-β, and TNF-β. Interferon-γ, the signature Th1 cytokine, acts directly on macrophages to enhance their microbiocidal and phagocytic activities. As a result, the activated macrophages can efficiently destroy intracellular pathogens and tumor cells. The Th2 cells produce IL-4, IL-5, IL-6, IL-10 and IL-13, which act by helping B cells develop into antibody-producing cells. Taken together, Th1 cells are primarily responsible for cell-mediated immunity, while Th2 cells are responsible for humoral immunity.

IL-18, the encoding nucleotide sequence and certain physicochemical chemical properties of the purified protein is known.

Kabushiki Kaisha Hayashibara Seibutsu Kayaku Kenkyujo's (“Hayashibara”), U.S. Pat. No. 5,912,324, which corresponds to EP 0 692 536 published on Jan. 17, 1996, discloses a mouse protein which induces IFN-gamma production by immunocompetent cells, the protein being further characterized as having certain physicochemical properties and a defined partial amino acid sequence. Also disclosed is a protein having a 157 aa sequence, two fragments thereof, DNA (471 bp) encoding the protein, hybridomas, protein purification methods, and methods for detecting the protein.

Hayashibara's U.S. Pat. No. 6,214,584, which corresponds to EP 0 712 931 published on May 22, 1996, discloses a 157 aa human protein and homologues thereof, DNA encoding the protein, transformants, processes for preparing the protein, monoclonal antibodies against the protein, hybridomas, protein purification methods, methods for detecting the protein, and methods of treatment and/or prevention of malignant tumors, viral diseases, bacterial infectious diseases, and immune diseases.

Incyte Pharmaceuticals, Inc.'s, WO 97/24441, published on Jul. 10, 1997, discloses a 193 aa protein corresponding to IL-18 precursor and encoding DNA.

Inflammatory bowel disease (IBD) is a group of chronic disorders that cause inflammation in the small and large intestine. IBD includes Crohn's disease and ulcerative colitis. Further, IBD can also include inflammatory colitis caused by bacteria, ischemia, radiation, drugs or chemical substances.

The present invention relates to the use of a IL-18 polypeptide for the treatment or prevention of IBD, including, but not limited to Crohn's disease, ulcerative colitis, and inflammatory colitis caused by bacteria, ischemia, radiation, drugs or chemical substances.

### SUMMARY OF THE INVENTION

In one aspect, the present invention provides a method of treating or preventing IBD, including, but not limited to Crohn's disease, ulcerative colitis, and inflammatory colitis caused by bacteria, ischemia, radiation, drugs or chemical substances; comprising, administering a therapeutically effective amount of a IL-18 polypeptide.

In further aspect, the invention also relates to a pharmaceutical composition comprising therapeutically effective amount of a IL-18 polypeptide to treat or prevent IBD, including, but not limited to Crohn's disease, ulcerative colitis, and inflammatory colitis caused by bacteria, ischemia, radiation, drugs or chemical substances, and a pharmaceutically acceptable carrier.

Yet in a further aspect, the present invention relates to the use of a IL-18 polypeptide in the preparation of a medicament for the treatment or prevention of IBD, including, but not limited to Crohn's disease, ulcerative colitis, and inflammatory colitis caused by bacteria, ischemia, radiation, drugs or chemical substances.

### DETAILED DESCRIPTION OF THE INVENTION

The following definitions are provided to facilitate understanding of certain terms and abbreviations used frequently in this application.

“Identity,” as known in the art, is a relationship between two or more polypeptide sequences or two or more polynucleotide sequences, as determined by comparing ihe sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, PROTEINS—STRUCTURE AND MOLECULAR PROPERTIES, 2nd Ed., T. E. Creighton, W. H. Freeman and Company, New York, 1993; Wold, F., Post-translational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182: 626-646 and Rattan et al., “Protein Synthesis: Post-translational Modifications and Aging”, Ann NY Acad Sci (1992) 663: 48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

Preferred parameters for polypeptide sequence comparison include the following:
1) Algorithm: Needleman and Wunsch, J. Mol. Biol. 48: 443-453 (1970) Comparison matrix: BLOSSUM62 from Hentikoff and Hentikoff, Proc. Natl. Acad. Sci. USA. 89: 10915-10919 (1992)
Gap Penalty: 12
Gap Length Penalty: 4

A program useful with these parameters is publicly available as the “gap” program from Genetics Computer Group, Madison Wis. The aforementioned parameters are the default parameters for peptide comparisons (along with no penalty for end gaps).

A polypeptide sequence of the present invention may be identical to the reference sequence of SEQ ID NO:1 or SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:1 or SEQ ID NO:2 by the numerical percent of the respective percent identity (divided by 100) and then subtracting that product from said total number of amino acids in SEQ ID NO:1 or SEQ ID NO:2, respectively, or:
nₐ≦xₐ−(xₐ·y)
wherein nₐ is the number of amino acid alterations, xₐ is the total number of amino acids in SEQ ID NO:1 or SEQ ID NO:2, and y is, for instance 0.70 for 70%, 0.80 for 80%, 0.85 for 85% etc., and wherein any non-integer product of xₐ and y is rounded down to the nearest integer prior to subtracting it from xₐ.

“Fusion protein” refers to a protein encoded by two, often unrelated, fused genes or fragments thereof. In one example, EP-A-0 464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

IL-18 Polypeptide

A IL-18 polypeptide is disclosed in EP 0692536A2, EP 0712931A2, EP0767178A1, and WO 97/2441. The polypeptides include isolated polypeptides comprising an amino acid sequence which has at least 70% identity, preferably at least 80% identity, more preferably at least 90% identity, yet more preferably at least 95% identity, most preferably at least 97-99% identity, to that of SEQ ID NO:1 (human IL-18) and SEQ ID NO:2 (murine IL-18) over the entire length of SEQ ID NO:1 and SEQ ID NO:2, respectively. Such polypeptides include those comprising the amino acid of SEQ ID NO:1 and SEQ ID NO:2, respectively.

Polypeptides of the present invention are interferon-γ-inducing polypeptides. They play a primary role in the induction of cell-mediate immunity, including induction of interferon-γ production by T cells and spleenocytes enhancement of the killing activity of NK cells and promotion of the differentiation of naive CD4+ T cells into Th1 cells. These properties are hereinafter referred to as “IL-18 activity” or “IL-18 polypeptide activity” or “biological activity of IL-18”. Also included amongst these activities are antigenic and immunogenic activities of said IL-18 polypeptides, in particular the antigenic and immunogenic activities of the polypeptides of SEQ ID NO:1 and SEQ ID NO:2. Preferably, a polypeptide of the present invention exhibits at least one biological activity of IL-18.

The polypeptides of the present invention may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

The present invention also includes variants of the aforementioned polypeptides, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

Polypeptides of the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

Recombinant polypeptides of the present invention may be prepared by processes well known in the art from genetically engineered host cells comprising expression systems. Accordingly, in a further aspect, the present invention relates to expression systems which comprises a polynucleotide or polynucleotides encoding the polypeptides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook et al., Molecular Cloning: A Laboratory Manual, 2nd Ed., Cold Spring Harbor Laboratory Press, Cold Spring Harbor, N.Y. (1989). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, high performance liquid chromatography, hydroxylapatite chromatography and lectin chromatography. Most preferably, affinity chromatography is employed for purification. Well-known techniques for refolding proteins may be employed to regenerate active conformation when the polypeptide is denatured during isolation and or purification.

The present invention also provides for pharmaceutical compositions comprising a therapeutically effective amount of IL-18, optionally in combination with another agent. Pharmaceutically acceptable carriers or excipients may also be employed. The pharmaceutical carrier employed may be, for example, either a solid or a liquid. Exemplary of solid carriers include, but are not limited to lactose, terra alba, sucrose, talc, gelatin, agar, pectin, acacia, magnesium stearate, stearic acid and the like. Exemplary of liquid carriers include, but are not limited to, saline, buffered saline, dextrose, water, glycerol, ethanol syrup, peanut oil olive oil, and combinations thereof. Similarly, the carrier or diluent may include time delay material well known in the art, such as glyceryl monostearate or glyceryl distearate alone or with a wax ethylcellulose, hydroxypropylmethylcellulose, methylmethacrylate and the like.

The invention further relates to pharmaceutical packs and kits comprising one or more containers filled with one or more of the ingredients of the aforementioned compositions of the invention. The polypeptides may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. In addition, if the present invention can be formulated in an enteric or an encapsulated formulation, oral administration may be possible. Alternative means for systemic administration include transmucosal and transdermal administration using penetrants such as bile salts or fusidic acids or other detergents. Administration of these combinations may also be topical and/or localized, in the form of salves, pastes, gels, and the like.

The dosage range of IL-18 required depends on the choice of adjuvant, if any, the route of administration, the nature of the formulation, the nature of the subject's condition, and the judgment of the attending practitioner. Suitable dosages of the composition, however, for IL-18 are in the range of 1 nanogram/kilogram to 1 milligram/kilogram of subject. Wide variations in the needed dosage, however, are to be expected in view of the variety of compounds available and the differing efficiencies of various routes of administration. For example, transdermal administration would be expected to require higher dosages than administration by intravenous injection. Variations in these dosage levels can be adjusted using standard empirical routines for optimization, as is well understood in the art.

The schedule for the administration of the composition depends on the dosage, on the choice of adjuvant, the route of administration, the nature of the formulation, the nature of the subject's condition, and the judgment of the attending practitioner. Suitable schedules for administration, are daily, weekly, or monthly. Wide variations in the schedules for the administration of the composition, however, are to be expected in view of the variety of other agents available and the differing efficiencies of various routes of administration. For example, transdermal administration would be expected to require higher dosages than administration by intravenous injection. Variations in these schedules for the administration of the composition can be adjusted using standard empirical routines for optimization, as is well understood in the art.

All publications, including but not limited to patents and patent applications, cited in this specification are herein incorporated by reference as if each individual publication were specifically and individually indicated to be incorporated by reference herein as though fully set forth.

It is believed that one skilled in the art can, using the preceding description, utilize the present invention to its fullest extent. Therefore the Examples herein are to be construed as merely illustrative and not a limitation of the scope of the present invention in any way.

### EXAMPLES

### Method

Induction of Colitis

Female, 8-week-old BALB/c mice (Charles River Japan) were used in this study. Colitis was induced by providing drinking water containing 3% dextran sulfate sodium (DSS, ICN Biomedicals Inc., M.W.=36,000-50,000) for 5 days. The administration of DSS was discontinued on day 5, and mice were given tap water alone for 7 days until on day 12.

Evaluation of Colitis

The disease activity index (DAI) was determined in all animals, by scoring body weight, stool consistency and rectal bleeding as described by Murthy, S. N. S. (Digestive Diseases and Sciences, 38(9) p. 1722-1734(1993)). The method of scoring is shown in Table 1. Severity of colitis was evaluated by area under the curve (AUC) calculated based on DAI curve ranged from day 3 to day 7 (AUC (3-7 day)), from day 7 to day 10 (AUC (7-10 day)), from day 10 to day 12 (AUC (10-12 day)) and from day 0 to day 12 (AUC (0-12 day)).

TABLE 1
Criteria for scoring

| | | | Occult blood or |
|---|---|---|---|
| Score | Weight loss (%) | Stool consistency | gross bleeding |
| 0 | None | Normal | Negative |
| 1 | 1-5 | Loose stool | Negative |
| 2 | 5-10 | Severe loose stool | Hemoccult positive |
| 3 | 10-15 | Diarrhea | Hemoccult strong positive |
| 4 | >15 | Severe diarrhea | Gross bleeding |

DAI = (combined score of weight loss, stool consistency and bleeding)/3.

### Experimental Design

Twelve mice were used in each group. IL-18 (SEQ ID NO: 2) was dissolved in buffer (25 mM Na-acetate, 100 mM NaCl, 0.1 mM EDTA, 6.0%(w/v) sucrose, pH 5.5). IL-18 at 0.3 ug/head or buffer was administered intraperitoneally once a day for 12 days from day 0.

The experimental groups were set up as follows:
Control*
3% DSS+buffer
3% DSS+IL-18 (0.3 ug/head)
* Mice which received tap water without DSS.

### Results

The effect of IL-18 on DSS-Induced Colitis

The effect of IL-18 on DSS-colitis was shown in Table 2. IL-18 (0.3 ug/head, i.p. q.d.) suppressed the severity of DSS-induced colitis as expressed by a significantly lower AUC (7-10 day), AUC (10-12 day) and AUC (0-12 day) compared with buffer-treated DSS-fed mice.

TABLE 2
Evaluation of colitis by AUC

| Groups | n | AUC (3-7 day) | AUC (7-10 day) | AUC (10-12 day) | AUC (0-12 day) |
|---|---|---|---|---|---|
| Control | 12 | 0.25 +/− 0.13 | 0.33 +/− 0.15 | 0.13 +/− 0.06 | 0.79 +/− 0.33 |
| 3% DSS + buffer | 11 | 5.36 +/− 0.53 | 7.38 +/− 0.54 | 4.32 +/− 0.52 | 18.21 +/− 1.52 |
| 3% DSS + IL-18 | 12 | 4.61 +/− 0.41 | 5.25 +/− 0.63** | 2.25 +/− 0.52** | 12.99 +/− 1.42* |
| Inhibition (%) | | (14.0) | (28.8) | (47.9) | (28.7) |

The data were represented as mean +/− SE.
n = 11 to 12.

TABLE 3
Sequence ID NO:1

```
Tyr Phe Gly Lys Leu Glu Ser Lys Leu Ser Val Ile Arg Asn Leu Asn
    1 5 10 15
Asp Gln Val Leu Phe Ile Asp Gln Gly Asn Arg Pro Leu Phe Glu Asp
                          20 25 30
Met Thr Asp Ser Asp Cys Arg Asp Asn Ala Pro Arg Thr Ile Phe Ile
                  35 40 45
Ile Ser Met Tyr Lys Asp Ser Gln Pro Arg Gly Met Ala Val Thr Ile
          50 55 60
Ser Val Lys Cys Glu Lys Ile Ser Thr Leu Ser Cys Glu Asn Lys Ile
65 70 75 80
Ile Ser Phe Lys Glu Met Asn Pro Pro Asp Asn Ile Lys Asp Thr Lys
                                  85 90 95
Ser Asp Ile Ile Phe Phe Gln Arg Ser Val Pro Gly His Asp Asn Lys
                        100 105 110
Met Gln Phe Glu Ser Ser Ser Tyr Glu Gly Tyr Phe Leu Ala Cys Glu
                115 120 125
Lys Glu Arg Asp Leu Phe Lys Leu Ile Leu Lys Lys Glu Asp Glu Leu
        130 135 140
Gly Asp Arg Ser Ile Met Phe Thr Val Gln Asn Glu Asp
145 150 155
```

TABLE 4
Sequence ID NO:2

```
Asn Phe Gly Arg Leu His Cys Thr Thr Ala Val Ile Arg Asn Ile Asn
    1 5 10 15
Asp Gln Val Leu Phe Val Asp Lys Arg Gln Pro Val Phe Glu Asp Met
                          20 25 30
Thr Asp Ile Asp Gln Ser Ala Ser Glu Pro Gln Thr Arg Leu Ile Ile
                  35 40 45
Tyr Met Tyr Lys Asp Ser Glu Val Arg Gly Leu Ala Val Thr Leu Ser
          50 55 60
Val Lys Asp Ser Lys Met Ser Thr Leu Ser Cys Lys Asn Lys Ile Ile
  65 70 75 80
Ser Phe Glu Glu Met Asp Pro Pro Glu Asn Ile Asp Asp Ile Gln Ser
                                  85 90 95
Asp Leu Ile Phe Phe Gln Lys Arg Val Pro Gly His Asn Lys Met Glu
                        100 105 110
Phe Glu Ser Ser Leu Tyr Glu Gly His Phe Leu Ala Cys Gln Lys Glu
                115 120 125
Asp Asp Ala Phe Lys Leu Ile Leu Lys Lys Lys Asp Glu Asn Gly Asp
        130 135 140
Lys Ser Val Met Phe Thr Leu Thr Asn Leu His Gln Ser
145 150 155
```

1. A method of treating or preventing IBD; comprising, administering a therapeutically effective amount of a polypeptide having at least 90% identity to the amino acid sequence of SEQ ID NO:1 over the entire length of SEQ ID NO:1.

2. A method of treating or preventing IBD; comprising, administering a therapeutically effective amount of a polypeptide having at least 90% identity to the amino acid sequence of SEQ ID NO:2 over the entire length of SEQ ID NO:2.

3. A method of treating or preventing IBD; comprising, administering a therapeutically effective amount of a polypeptide having the amino acid sequence of SEQ ID NO:1.

4. A method of treating or preventing IBD; comprising, administering a therapeutically effective amount of a polypeptide having the amino acid sequence of SEQ ID NO:2.

5. A method of claim 1, 2, 3 or 4 in which IBD is selected from the group consisting of Crohn's disease, ulcerative colitis, and inflammatory colitis caused by bacteria, ischemia, radiation, drugs or chemical substances.

6. A pharmaceutical composition for treating or preventing IBD comprising therapeutically effective amount of a polypeptide having at least 90% identity to the amino acid sequence of SEQ ID NO:1 over the entire length of SEQ ID NO:1.

7. A pharmaceutical composition for treating or preventing IBD comprising therapeutically effective amount of a polypeptide having at least 90% identity to the amino acid sequence of SEQ ID NO:2 over the entire length of SEQ ID NO:2.

8. A pharmaceutical composition for treating or preventing IBD comprising therapeutically effective amount of a polypeptide having the amino acid sequence of SEQ ID NO:1.

9. A pharmaceutical composition of claims 6-8 in which IBD is selected from the group consisting of Crohn's disease, ulcerative colitis, and inflammatory colitis caused by bacteria, ischemia, radiation, drugs or chemical substances.
